(19) United States
(12) Patent Application Publication
(10) Pub. No.: US 2010/0298601 A1
(43) Pub. Date: Nov. 25, 2010

(54) Catalyst System, Oxidation Reactor Containing The Same, And Preparation Method For Acrolein And Acrylic Acid Using The Same

(75) Inventors: Byung-Yul Choi, Jeollanam-do (KR); Hyun-Jong Shin, Gwangju Metropolitan City (KR); Young-Hyun Choe, Jeollanam-do (KR); Young-Jin Cho, Jeollanam-do (KR); Duk-Ki Kim, Gwangju Metropolitan City (KR); Ju-Yeon Park, Gwangju Metropolitan City (KR)

Correspondence Address: LGCHEM;Lerner, David, Littenberg, Krumholz & Mentlik, LLP, 600 South Avenue West, Westfield, NJ, 07090, US

(73) Assignee: LG CHEM, LTD., Seoul (KR)

(21) Appl. No.: 12/812,803

(22) PCT Filed: Jan. 16, 2009
(86) PCT No.: PCT/KR2009/000245

§ 371 Date: Jul. 14, 2010

(30) Foreign Application Priority Data
Jan. 17, 2008 (KR) 10-2008-0005383

(51) Int. Cl. B01J 23/88; B01J 8/06; B01J 8/04; C07C 45/35; C07C 51/235

(52) U.S. Cl. 562/534; 502/311; 422/190; 568/449; 568/479

(57) ABSTRACT

The present invention relates to a catalyst system, an oxidation reactor comprising the same, and a method for producing an acrolein and an acrylic acid by using the same. By using the catalyst system according to the present invention, when acrolein and acrylic acid are produced, since heat accumulation in a catalyst layer may be effectively prevented, catalyst deterioration may be prevented, and the catalyst may be stably used for a long period of time. In addition, an acrolein and an acrylic acid may be produced at high selectivity and high yield.

### TECHNICAL FIELD

The present invention relates to a catalyst system, an oxidation reactor comprising the same, and a method for producing an acrolein and an acrylic acid by using the same. More particularly, the present invention relates to a method for producing an acrolein and an acrylic acid by oxidizing propylene under a catalyst system that comprises catalyst particles having different activities.

This application claims priority from Korean Patent Application No. 2008-0005383 filed on Jan. 17, 2008 in the KIPO, the disclosure of which is incorporated herein by reference in its entirety.

### BACKGROUND ART

By using a shell-and-tube heat exchange type of fixed layer multitube reactor, under the Mo—Bi (molybdenum-bismuth containing) oxidation catalyst, the oxidation of propylene by a gas phase contact reaction using oxygen molecules or oxygen molecule-containing gas has been widely carried out in the industry.

Since the gas phase oxidation reaction of propylene is a severe exothermic reaction, a hot spot is easily formed in the catalyst layer of each reaction tube. The occurrence of over-hot spot causes over-oxidation, and the yields of acrolein and acrylic acid are reduced. In addition, the occurrence of over heat in the hot spot of the catalyst layer deteriorates the catalyst, and makes it impossible to stably carry out the oxidation reaction for a long period of time. In particular, at an inlet side of raw material in the reactor, in the case of when the concentration of propylene is increased, or in the case of when the space velocity of the raw material is increased in order to increase the productivity, a problem of hot spot is watched with keen interest. Accordingly, in order to suppress the occurrence of the above hot spot, many methods have been proposed.

In general, in a catalyst reaction that is accompanied with heat emission, as a method for effectively controlling over heat of a hot spot portion, various methods such as a method for reducing a space velocity by reducing an amount of feed gas, a method for using a reaction tube having a small inner diameter and the like are known. However, if the space velocity is reduced, it is disadvantageous in terms of high productivity in the industry, and the method for reducing the inner diameter of the reaction tube is disadvantageous in that there is a difficulty in production of the reactor, there is an economic disadvantage in respects to production cost of the reactor, and many efforts and more time are required to fill the catalyst. Therefore, in the industrial process, a method for avoiding the above methods, maintaining the high yield and the high productivity, and stably using the catalyst for a long period of time is necessarily required, and many studies have been made of this.

For example, when the catalyst is filled, various methods for solving the problems, such as a technology for filling the catalyst so that the volume of the catalyst is sequentially reduced from an inlet side of raw gas to an outlet side of raw gas (KR 1995-0004027), a technology for filling the catalyst so that the activity of the catalyst is increased from an inlet side of raw gas to an outlet side of raw gas (KR 0487883), a technology for filling an inactive shaped body in an inlet side of raw gas of a reaction tube to suppress heat accumulation around a hot spot (Japanese Examined Patent Application publication No. 53-30688) and the like, are proposed. However, the above methods for minimizing catalyst deterioration or side reactions by reducing the temperature of hot spot are not effective to solve the above-mentioned problems.

In the case of a method in which several catalysts having different occupation volumes are manufactured and the catalysts having small occupation volumes are continuously filled in continuous reaction tubes manufactured from an inlet side of raw gas to an outlet side of raw gas, the occupation volume of the catalyst is limited by the diameter of each reaction unit, and it is difficult to fill several desired catalysts in the reaction unit.

In addition, in the several catalysts that show different activity levels, since the contents of the specific components in the catalysts, which are to be controlled, are smaller than those of other components, it is impossible to manufacture it with excellent reproducibility. As a method for increasing the activity from an inlet side to an outlet side, controlling of the activity level by calcination does not have excellent reproducibility because the inner temperature distribution of the oven that is used in the calcination. In particular, such is the case of when the catalysts having different activity levels are manufactured in a great amount.

Therefore, the above methods do not completely satisfy the suppression of the occurrence of hot spot.

In addition, in the practical industry, instead of the above methods that have a problem in catalyst manufacturing or do not sufficiently satisfy solving of hot spot problems, a method for easily mixing an introduction side of reaction starting gas at a front of a catalyst layer with an inactive shaped body and diluting them has been widely used.

However, as described above, in the method for performing filling by diluting using the inactive shaped body, if the catalyst filling amounts (effective component amount/total amount) according to the area are different from each other for each reaction tube, there are problems in that the temperature of the catalyst layer largely varies for each reaction tube, and the yield or the reaction ratio largely varies. Because of the nonuniformity thusly generated, the yield in respects to the total reactor and the reaction ratio are reduced, and the reaction is not uniform in respects to the entire reactor, thus the it is impossible to sufficiently improve productivity.

To make the reaction state of each reaction tube uniform in the oxidation reactor is important in views of stable operation of the oxidation reactor. Under the optimumly selected condition with the assumption of uniform, an excessive reaction may occur, side reactions may be increased, and the selectivity may be reduced in the tube in which local deviation occurs. In some cases, the temperature of the overhot spot may be rapidly increased to the temperature that is not capable of being locally controlled or more. Since the reaction states are different from each other for each reaction tube, the catalyst deterioration states are different from each other, and the total catalyst life span is reduced.

Accordingly, there remains a need to develop a technology regarding a method for minimizing catalyst deterioration and side reactions because of severe heat emission at an overhot spot generated by the catalyst reaction and stably maintaining the high productivity for a long period of time.

### DISCLOSURE

### Technical Problem

Therefore, it is an object of the present invention to provide an industrially advantageous method for producing an acrolein and an acrylic acid. More particularly, it is an object of the present invention to provide a method that is capable of producing an acrolein and an acrylic acid at high yield by more effectively suppressing the occurrence of hot spot in a reaction area or heat accumulation in the hot spot to extend a life span of the catalyst.

### Technical Solution

Therefore, the present invention provides a catalyst system which comprises 1) a complex catalyst particle that is obtained by shaping a mixture of a catalyst effective component material and an inactive material; and 2) a pure catalyst particle that is obtained by shaping a catalyst effective component material.

In addition, the present invention provides an oxidation reactor that comprises the catalyst system.

In addition, the present invention provides a method for producing an acrolein and an acrylic acid by using the catalyst system.

### ADVANTAGEOUS EFFECTS

A catalyst system according to the present invention may show an uniform performance ability because components of a catalyst are uniformly disposed in an axis direction from an inlet side to an outlet side for each reaction tube. Therefore, the occurrence of hot spot in a catalyst layer in which catalyst particles are filled or heat accumulation in the hot spot may be effectively prevented, deterioration of the catalyst may be prevented, and the catalyst may be stably used for a long period of time. In addition, by using the catalyst system according to the present invention, an acrolein and an acrylic acid may be produced at high selectivity and high yield.

### BEST MODE

Hereinafter, the present invention will be described in detail.

A catalyst system according to the present invention comprises 1) a complex catalyst particle that is obtained by shaping a mixture of a catalyst effective component material and an inactive material; and 2) a pure catalyst particle that is obtained by shaping a catalyst effective component material.

In the catalyst system according to the present invention, it is preferable that the catalyst effective component material in 1) the complex catalyst particle and 2) the pure catalyst particle are a metal oxide that is represented by the following Formula 1.

Moₐ A_{b} B_{c} C_{d} Dₑ E_{f} F_{g} Oₕ (Formula 1)

wherein Mo is molybdenum,

A is one or more elements that are selected from the group consisting of Bi and Cr,

B is one or more elements that are selected from the group consisting of Fe, Zn, Mn, Nb, and Te,

C is one or more elements that are selected from the group consisting of Co, Rh, and Ni,

D is one or more elements that are selected from the group consisting of W, Si, Al, Zr, Ti, Cr, Ag, and Sn,

E is one or more elements that are selected from the group consisting of P, Te, As, B, Sb, Sn, Nb, Cr, Mn, Zn, Ce, and Pb,

F is one or more elements that are selected from the group consisting of Na, K, Li, Rb, Cs, Ta, Ca, Mg, Sr, Ba, and MgO,

a, b, c, d, e, f, and g are an atomic ratio of each element,

when a=10, b is in the range of 0.01 to 10, c is in the range of 0.01 to 10, d is in the range of 0 to 10, e is in the range of 0 to 10, f is in the range of 0 to 20, g is in the range of 0 to 10, and h is a value that is determined according to an oxidation state of each component.

The shape of 1) the complex catalyst particle or 2) the pure catalyst particle may be a cylinder shape or a hollow cylinder shape, but is not limited thereto. The shape may be a sphere shape, a cylindroid shape (pelletization) or a ring shape. It is not required that the sphere shape is a complete sphere shape, but it is enough if the particle is substantially sphere. The same goes for the cases of the cylinder shape or ring shape.

The outer diameter of 1) the complex catalyst particle or 2) the pure catalyst particle is in the range of preferably 3 to 10 mm, and more preferably 5 to 8 mm. In addition, the ratio (L/D) of the length and the diameter (outer diameter) of 1) the complex catalyst particle or 2) the pure catalyst particle is in the range of preferably 1 to 1.3, and it is more preferable that L/D=1.

Here, the outer diameter means a diameter of an outer circle of a donut shape of section in the case of when the shape of the particle is the hollow cylinder shape. In addition, the length means a length between both ends in an axis direction of the particle in the case of when the shape of the particle is the cylinder shape or the hollow cylinder shape. In addition, the diameter means a diameter of the circular section passing through the center thereof in the case of when the shape of the particle is the sphere shape and a diameter of the circle section in the case of when the shape of the particle is the cylinder shape.

1) the complex catalyst particle or 2) the pure catalyst particle may be used by itself, or may be used as a catalyst particle that is supported in a carrier that is generally used, such as α-alumina, silicon carbide, axinite, silica, zirconium oxide, and titanium oxide.

In the catalyst system according to the present invention, the content of the inactive material in 1) the complex catalyst particle may vary according to the number of catalyst layers in which the catalyst particles are filled, but it is preferable that the content is in the range of 20 to 80 vol %. In the case of when the content of the inactive material is less than 20 vol %, it is difficult to effectively control the over hot spot that may be formed in the catalyst layer in which the complex catalyst particle is filled, and in the case of when the content is more than 80 vol %, the amount of the catalyst effective component is too small, it is difficult to act as the catalyst layer. Thus, in terms of the productivity using the catalyst system, it may be noneffective.

The inactive material means an inactive material that is used in an oxidation reaction for producing acrolein and acrylic acid from propylene and the like, examples of the inactive material include silica, alumina, silica alumina, zirconium oxide, titanium oxide and the like, and one or more thereof may be used while being mixed with each other.

The inactive material may exist in a granule shape or a powder shape. The granule shape means the degree of discrimination of the shape by the naked eye, the size of the granule shape is ½ or less of the size of the final shaped catalyst, and the granule shape may have the size that is useful to produce the shaped catalyst having the size in the range of 0.1 to 2 mm. In addition, the powder shape means a fine powder, that is, a powder material having less than the minimum size of the granule shape. The powder shape is advantageous in that it is easy to obtain the powder shape, and it is obtained at low cost by pulverizing the dried material, and the granule shape has an advantage in that handling is easy as compared to the powder shape. Thus, in a practical industrial process, the inactive material may be appropriately selected from the granule shape and the powder shape according to the condition.

Here, the granule means a particle that has the particle size of at least 0.1 mm or more, and the powder means a fine powder that has the size of less than 0.1 mm.

In the catalyst system according to the present invention, 1) the complex catalyst particle may be produced by sequentially mixing starting raw materials that constitute the catalyst, for example, the catalyst effective component material that are represented by Formula 1 and the inactive material, with each other in water, producing an aqueous solution or an aqueous slurry, and carrying out processes such as drying, shaping, and sintering. In addition, 2) the pure catalyst particle may be produced by using the same method as the production method of the complex catalyst particle, except that the inactive material is excluded but the catalyst effective component material is used as the starting raw material.

Here, 2) the pure catalyst particle that is obtained by shaping the catalyst effective component material is not produced by mixing the catalyst effective component material with the inactive material in each shaping particle unit to shape the final mixture, but means a particle that is produced by forming the final shaped particle using only the effective component material substantially acting as the catalyst. The material that substantially acting as the catalyst means that in order to produce a body that has a physically predetermined shape and size and is filled in a commercially used plant and used, it includes almost only the catalyst effective component material with the exception of various shaping additives that are used in a middle process, for example, a shaping aiding agent, a reinforcing agent and/or a pore forming agent, remain in a small amount in the final shaped body. For example, this means that it includes 95 to 100% of the catalyst effective component material.

In addition, various materials that are used produced for a specific purpose in the course of producing the catalyst, such as the shaping aiding agent that is capable of improving the shapability, the reinforcing agent that is capable of improving the strength of the catalyst, a pore forming agent that is capable of providing a predetermined pore to the catalyst and the like, when 1) the complex catalyst particle or 2) the pure catalyst particle are produced, may be added thereto.

Examples of this material may include a stearinic acid, a maleic acid, ammonium nitrate, ammonium carbonate, graphite, starch, cellulose, glass fiber and the like, but are not limited thereto. It is preferable that the addition of the materials does not negatively affect performance of the catalyst. In particular, in the case of when the addition amount of the materials is excessive, since mechanical strength may be significantly reduced, it is preferable that the amount to prevent the mechanical strength of the catalyst from being reduced to the degree of non-practicality is added thereto.

In the art, the commercially used supported catalyst is carried produced by coating an active component on the shaped inactive carrier material, that is, the shaped carrier, in various manners and forming an active component coated layer on a shell of the inactive carrier. However, when the complex catalyst particle according to the present invention is compared to a catalyst that is produced by using a known general method, they are different from each other in views of the production method and product characteristics of the finally obtained product, for example, the internal structure of each shaped particle and the internal distribution of constitution materials.

A known carried catalyst is produced by after preshaping material that is an inactive carrier or purchasing pre-shaped products, coating a separately produced active component slurry or powder after drying or sintering thereon. On the other hand, in order to perform differentiated filling of the present invention, the complex catalyst that is produced to fill in a front side of the catalyst layer is basically different from a known carried catalyst in that in the course of producing the inactive material (powder shape, granule shape and the like) and the effective component powder, mixing is performed to produce a uniform mixture at a predetermined time such as when it exists in a liquid phase, or in a powder state, and the final shaping step is then performed to produce the catalyst.

Therefore, the catalyst system according to the present invention has an advantage in that the reproducibility and the regenerability are particularly easy and it is very suitable to mass production. That is, as the catalyst that shows the uniform performance ability, there is an advantage of mass production.

In the case of when two or more catalyst layers are filled in the reactor by using the catalyst system according to the present invention, the two or more catalyst layers may comprise the first catalyst layer in which 1) the complex catalyst particle is filled, and the second catalyst layer in which 2) the pure catalyst particle is filled, but are not limited thereto.

In addition, the first catalyst layer is classified into two or more layers, and may be two or more catalyst layers in which the complex catalyst particles that have different content ratios of the catalyst effective component material and the inactive material are filled in separate catalyst layers.

The number of the catalyst layers in which the complex catalyst particles that has different content ratios of the catalyst effective component material and the inactive material are filled is not particularly limited, but it is preferable that the number of the catalyst layers is 2 or 3 in views of industry.

In addition, in the catalyst layer, a partition ratio of the catalyst layer, which is selected a relative length of each reaction area to a total length of a reaction tube, may be appropriately selected so as to obtain the optimum activity and selectivity according to an oxidation reaction condition, or the composition, the shape, and the size of the catalyst that is filled in each catalyst layer.

In addition, the present invention provides an oxidation reactor which comprises the catalyst system, and in which a first catalyst layer in which 1) the complex catalyst particle is filled is disposed at an inlet side of a raw material in a reactor, and a second catalyst layer in which 2) the pure catalyst particle is filled is disposed at an outlet side of a raw material in the reactor.

The oxidation reactor according to the present invention is characterized in that at an inlet side of the raw material, in order to prevent formation of overhot spot in the catalyst layer, the complex catalyst particle that is controlled by introducing the inactive material so as to reduce the activity is filled in the catalyst layer, and at an outlet side of the reactor, the pure catalyst particle having the high activity, from which the inactive material is excluded, is filled in the catalyst layer.

It is preferable that the oxidation reactor is a shell-and-tube heat exchange type of fixed layer multitube reactor, but is not limited thereto.

In addition, the present invention provides a method for producing an acrolein, which comprises the step of performing a fixed layer catalyst partial oxidation reaction to propylene by using the oxidation reactor.

In addition, the present invention provides a method for producing an acrylic acid, which comprises the steps of a) using the oxidation reactor and performing a fixed layer catalyst partial oxidation reaction to propylene to produce an acrolein; and b) performing a fixed layer catalyst partial oxidation reaction to the produced acrolein.

The method for producing the acrylic acid from propylene is generally carried out by two stage partial contact gas phase oxidation reaction. That is, in the first stage reaction area, propylene is oxidized by oxygen, diluted inactive gas, steam and a catalyst in a predetermined amount to mainly produce an acrolein, and in the second stage reaction area, the acrolein is oxidized by oxygen, diluted inactive gas, steam and a catalyst in a predetermined amount to produce the acrylic acid. In the first stage reaction area, since the produced acrolein is continuously oxidized, the acrylic acid may be partially generated.

The method for producing the acrolein and acrylic acid according to the present invention is characterized in that in the first stage reaction area comprise two or more catalyst layers, and the catalyst particles having different activities are filled in each catalyst layer.

In the method for producing acrolein and acrylic acid according to the present invention, in the gas pase partial oxidation reaction in which acrolein is mainly produced from propylene, the reaction temperature is in the range of 200 to 450° C., and preferably 200 to 370° C., and the reaction pressure is in the range of 0.1 to 10 atm, and preferably 0.5 to 3 atm.

In addition, the raw material for performing the reaction may comprise 5 to 10 vol % of propylene, 10 to 15 vol % of oxygen, 5 to 60 vol % of steam, and 20 to 80 vol % of inactive gas. Here, oxygen may be 13 vol %. In addition, by introducing the raw material in a space velocity of the raw material which is in the range of 500 to 5,000 hr⁻¹ (STP), the oxidation reaction may be carried out.

In the method for producing acrolein and acrylic acid according to the present invention, the catalyst particles that are filled in the two or more catalyst layers in the first stage reaction area are different from each other, the complex catalyst particle that is obtained by shaping the mixture of the catalyst effective component material and the inactive material is filled in the catalyst layer that is disposed at an inlet side of the raw material, and the pure catalyst particle that is obtained by shaping the catalyst effective component from which the inactive material is excluded is filled in the catalyst layer that is disposed at an outlet side of the reactor.

That is, by introducing the inactive material at an inlet side of the raw material in order to prevent formation of the overhot spot in the catalyst layer, the complex catalyst particle that is controlled to reduce the activity is filled in the catalyst layer, and at an outlet side of the reactor, the pure catalyst particle having the high activity, from which the inactive material is excluded, is filled in the catalyst layer.

In the method for producing acrolein and acrylic acid according to the present invention, the second stage reaction for mainly producing the acrylic acid from the acrolein may be carried out by introducing the mixture gas that comprises 1 to 10 vol % of acrolein as raw gas, 0.5 to 20 vol % of oxygen (molecular oxygen), 0 to 60 vol % of steam, and 20 to 80 vol % of inactive gas as diluted gas (for example, nitrogen, carbon gas and the like) at a temperature in the range of 200 to 400° C. and a space velocity in the range of 300 to 5,000 hr⁻¹ (STP) into each reaction tube, and contacting it with a Mo—V catalyst that is generally used in the second stage reaction to perform reaction.

In the method for producing acrolein and acrylic acid according to the present invention, it is preferable that the catalyst layer in the first stage reaction area comprises two layers, the complex catalyst particle that is obtained by shaping the mixture of the catalyst effective component material and the powder shape or the granule shape inactive material is filled in the catalyst layer that is disposed at an inlet side of the raw material, and the pure catalyst particle from which the inactive material is excluded and which is obtained by shaping the catalyst effective component material is filled in the catalyst layer that is disposed at an outlet side of the reactor.

In addition, in the method for producing acrolein and acrylic acid according to the present invention, it is more preferable that the catalyst layer in the first stage reaction area comprises three layers, the catalyst layer in which the complex catalyst particle that is obtained by shaping the mixture of the catalyst effective component material and the granule shape inactive material is filled is disposed at an inlet side of the raw material, the catalyst layer in which the complex catalyst particle that is obtained by shaping the mixture of the catalyst effective component material and the powder shape inactive material is filled is disposed next thereto, and the catalyst layer in which the pure catalyst particle from which the inactive material is excluded and which is obtained by shaping the catalyst effective component material is filled is disposed at an outlet side of the reactor.

### MODE FOR INVENTION

Hereinbelow, the present invention will be described in detail with reference to Examples. The present invention may, however, be embodied in many different forms and should not be construed as being limited to the Examples set forth herein.

### EXAMPLE

The present invention is illustrated in more detail by mentioning performation of Example, and here, conversion ratio, selectivity, and one penetration yield are as defined below:

propylene conversion ratio (mole %)=(the mole number of reacted propylene/the mole number of supplied propylene)×100

selectivity (mole %)=(the total mole number of the formed target product (acrolein or acrylic acid)/the mole number of reacted propylene)×100

one penetration yield (mole %)=(the total mole number of the formed acrolein and acrylic acid/the mole number of supplied propylene)×100

### Preparation Example 1

### Production of the Catalyst 1

While 2,500 Ml of distilled water was heated and agitated at 70 to 85° C., 1,000 g of molybdenum acid ammonium was dissolved to produce a solution 1. To 400 Ml of distilled water, 274 g of bismuth nitrate, 228 g of iron nitrate and 1.9 g of potassium nitrate were added, and well mixed with each other, and 71 g of nitric acid was added thereto and dissolved therein to produce a solution 2. In 200 Ml of distilled water, 618 g of cobalt nitrate was dissolved to produce a solution 3. After the solution 2 and the solution 3 were mixed with each other, while the temperature of the solution was maintained at 40 to 60° C., they were mixed with the solution 1 to produce a catalyst suspension solution.

After the produced suspension solution was dried to produce Mo₁₂Bi_{1.2}Fe_{1.2}Co_{4.5}K_{0.04}, and it was pulverized to 150 μm or less. After the pulverized catalyst powder was mixed for 2 hours, it was shaped into a cylinder shape. After the catalyst was shaped so that the outer diameter of the catalyst was in the range of 4.0 to 6.0 mm, it was sintered at 500° C. for 5 hours under an air atmosphere to produce a catalyst 1.

### Preparation Example 2

### Production of the Catalyst 2

While 2,500 Ml of distilled water was heated and agitated at 70 to 85° C., 1,000 g of molybdenum acid ammonium was dissolved to produce a solution 1. To 400 Ml of distilled water, 274 g of bismuth nitrate, 228 g of iron nitrate and 1.9 g of potassium nitrate were added, and well mixed with each other, and 71 g of nitric acid was added thereto and dissolved therein to produce a solution 2. In 200 Ml of distilled water, 618 g of cobalt nitrate was dissolved to produce a solution 3. After the solution 2 and the solution 3 were mixed with each other, while the temperature of the solution was maintained at 40 to 60° C., they were mixed with the solution 1 to produce a catalyst suspension solution. Alumina was added thereto as the inactive material powder in an amount that was ⅔ of the volume of the catalyst effective component after the drying, and agitated to be uniformly dispersed and mixed with each other.

The produced suspension solution was dried and the pulverized catalyst powder was mixed for 2 hours, it was shaped into a cylinder shape. After the catalyst was shaped so that the outer diameter of the catalyst was in the range of 4.0 to 6.0 mm, it was sintered at 500° C. for 5 hours under an air atmosphere to produce a catalyst 2.

### Preparation Example 3

### Production of the Catalyst 3

Like in Preparation Example 2, after the solution 2 and the solution 3 were mixed with each other, while the temperature of the solution was maintained at 40 to 60° C., there were mixed with the solution 1 to produce the catalyst suspension solution, and the catalyst 3 was produced by using the same method as Preparation Example 2, except that alumina that was added as the inactive material was added in a granule shape having an average diameter of about 2 mm instead of powder.

### Example 1

In the stainless steel reaction tube that was heated by melted nitrates and had the inner diameter of 25 mm, the catalyst 2 of Preparation Example 2 was filled from a gas inlet side to a gas outlet side so that the length of the catalyst layer was 1,000 mm, and the catalyst 1 of Preparation Example 1 was filled in a rear side so that the length of the catalyst layer was 2,000 mm. That is, in the reaction tube, the catalyst layer was separated into two reaction units, the catalyst 2 was filled in the reaction unit at the gas inlet side, and the catalyst 1 was filled in the reaction unit at the gas outlet side.

The oxidation reaction was carried out by introducing the raw gas of 7 vol % of propylene, 13 vol % of oxygen, 8 vol % of steam and 72 vol % of inactive gas at the reaction temperature of 310° C. under the reaction pressure of 0.7 atm at the space velocity of 1,400 hr⁻¹ (STP) into the catalyst. The results are described in the following Table 1.

### Example 2

The oxidation reaction was carried out by using the same method as Example 1, except that the catalyst 3 of Preparation Example 3 was used instead of the catalyst 2. The results are described in the following Table 1.

### Example 3

The oxidation reaction was carried out by using the same method as Example 1, except that while only the catalyst 2 was not filled in the gas introduction side of the reaction tube having the whole length of 1,000 mm in Example 1, the introduction side was separated into two layers, the catalyst 3 was filled in the portion of 500 mm and the catalyst 2 was filled in the rear portion of 500 mm. The results are described in the following Table 1.

### Comparative Example 1

In the stainless steel reaction tube that was heated by melted nitrates and had the inner diameter of 25 mm, the catalyst dilute substance in which alumina balls were mixed at a mixing ratio of 40 vol % as the inactive shaped body that had the same size as the catalyst 1 was filled from a gas inlet side to a gas outlet side so that the length of the catalyst layer was 1,000 mm, and the catalyst 1 was filled in a rear side so that the length of the catalyst layer was 2,000 mm. That is, in the reaction tube, the catalyst layer was separated into two reaction units, the dilute substance in which the catalyst 1 and the inactive shaped body were mixed was filled in the reaction unit at the gas inlet side, and only the catalyst 1 was filled in the reaction unit at the gas outlet side.

The oxidation reaction was carried out by introducing the raw gas of 7 vol % of propylene, 13 vol % of oxygen, 8 vol % of steam and 72 vol % of inactive gas at the reaction temperature of 310° C. under the reaction pressure of 0.7 atm at the space velocity of 1,400 hr⁻¹ (STP) into the catalyst.

TABLE 1

| | | | | | One | Overhot |
|---|---|---|---|---|---|---|
| | | | | (ACR + | pen- | spot |
| | Propylene | Selectivity | | AA) | etration | tem- |
| | conversion | (mole %) | | selectivity | yield | perature |
| | (mole %) | ACR | AA | (mole %) | (mole %) | (° C.) |
| Example 1 | 97.3 | 82.0 | 10.3 | 92.3 | 89.8 | 364 |
| Example 2 | 97.2 | 82.2 | 10.3 | 92.5 | 89.9 | 360 |
| Example 3 | 97.5 | 82.7 | 10.0 | 92.7 | 90.4 | 362 |
| Comparative | 97.3 | 77.6 | 12.3 | 89.9 | 87.5 | 373 |
| Example 1 | | | | | | |

ACR: acrolein,
AA: acrylic acid.

As described above, under the high load reaction condition such as the high raw material concentration condition or the high space velocity, the catalyst system according to the present invention allows the components of the catalyst to be uniformly disposed in an axis direction of the reaction tube from an inlet side to an outlet side for each reaction tube, thus showing a uniform performance ability.

Therefore, since it is effective to discharge and emit heat that is generated in the catalyst layer in which the catalyst particle are filled, the occurrence of hot spot or heat accumulation in hot spot may be effectively prevented, catalyst deterioration may be prevented, and the catalyst may be stably used for a long period of time.

In addition, if the catalyst system according to the present invention is used, it is very useful to produce acrolein and acrylic acid in an industrial scale and acrolein and acrylic acid may be produced at high selectivity and high yield.

1. A catalyst system comprising:
1) a complex catalyst particle that is obtained by shaping a mixture of a catalyst effective component material and an inactive material; and
2) a pure catalyst particle that is obtained by shaping a catalyst effective component material.

2. The catalyst system according to claim 1, wherein the catalyst effective component material in 1) the complex catalyst particle and 2) the pure catalyst particle are a metal oxide that is represented by the following Formula 1:
Moₐ A_{b} B_{c} C_{d} Dₑ E_{f} F_{g} Oₕ (Formula 1)
wherein Mo is molybdenum,
A is one or more elements that are selected from the group consisting of Bi and Cr,
B is one or more elements that are selected from the group consisting of Fe, Zn, Mn, Nb, and Te,
C is one or more elements that are selected from the group consisting of Co, Rh, and Ni,
D is one or more elements that are selected from the group consisting of W, Si, Al, Zr, Ti, Cr, Ag, and Sn,
E is one or more elements that are selected from the group consisting of P, Te, As, B, Sb, Sn, Nb, Cr, Mn, Zn, Ce, and Pb,
F is one or more elements that are selected from the group consisting of Na, K, Li, Rb, Cs, Ta, Ca, Mg, Sr, Ba, and MgO,
a, b, c, d, e, f, and g are an atomic ratio of each element, and
when a=10, b is in the range of 0.01 to 10, c is in the range of 0.01 to 10, d is in the range of 0 to 10, e is in the range of 0 to 10, f is in the range of 0 to 20, g is in the range of 0 to 10, and h is a value that is determined according to an oxidation state of each component.

3. The catalyst system according to claim 1, wherein the shape of 1) the complex catalyst particle or 2) the pure catalyst particle is selected from the group consisting of a cylinder shape or a hollow cylinder shape, a sphere shape, a cylindroid shape and a ring shape.

4. The catalyst system according to claim 1, wherein the outer diameter of 1) the complex catalyst particle or 2) the pure catalyst particle is in the range of 3 to 10 mm.

5. The catalyst system according to claim 1, wherein the ratio (L/D) of the length and the outer diameter of 1) the complex catalyst particle or 2) the pure catalyst particle is in the range of 1 to 1.3.

6. The catalyst system according to claim 1, wherein 1) the complex catalyst particle or 2) the pure catalyst particle is a catalyst particle that is supported in a carrier that is selected from the group consisting of α-alumina, silicon carbide, axinite, silica, zirconium oxide, and titanium oxide.

7. The catalyst system according to claim 1, wherein the content of the inactive material in 1) the complex catalyst particle is in the range of 20 to 80 vol %.

8. The catalyst system according to claim 1, wherein the inactive material in 1) the complex catalyst particle is selected from the group consisting of silica, alumina, silica alumina, zirconium oxide, and titanium oxide.

9. The catalyst system according to claim 1, wherein the shape of the inactive material in 1) the complex catalyst particle is a granule shape or a powder shape.

10. The catalyst system according to claim 1, wherein 1) the complex catalyst particle or 2) the pure catalyst particle further comprises one or more materials that are selected from the group consisting of a shaping aiding agent, a reinforcing agent, and a pore forming agent.

11. The catalyst system according to claim 1, wherein the catalyst system comprises a first catalyst layer that is formed by filling 1) the complex catalyst particle and a second catalyst layer that is formed by filling 2) the pure catalyst particle.

12. The catalyst system according to claim 11, wherein the first catalyst layer is classified into two or more catalyst layers, and in the two or more catalyst layers, complex catalyst particles that have different content ratios of the catalyst effective component material and the inactive material are filled.

13. An oxidation reactor comprising:
the catalyst system according to claim 1,
wherein a first catalyst layer in which the complex catalyst particle that is obtained by shaping a mixture of catalyst effective component material and the inactive material is filled is disposed at an inlet side of a raw material in a reactor, and a second catalyst layer in which the pure catalyst particle that is obtained by shaping the catalyst effective component material is filled is disposed at an outlet side of a raw material in the oxidation reactor.

14. The oxidation reactor according to claim 13, wherein the oxidation reactor is a shell-and-tube heat exchange type of fixed layer multitube reactor.

15. A method for producing an acrolein,
the method comprising the step of:
performing a fixed layer catalyst partial oxidation reaction to propylene by using the oxidation reactor of claim 13.

16. The method for producing an acrolein according to claim 15, wherein the step of performing a fixed layer catalyst partial oxidation reaction is carried out at a reaction temperature in the range of 200 to 450° C. and a reaction pressure in the range of 0.1 to 10 atm.

17. The method for producing an acrolein according to claim 15, wherein in order to carry out the oxidation reaction, a raw material comprising 5 to 10 vol % of propylene, 10 to 15 vol % of oxygen, 5 to 60 vol % of steam, and 20 to 80 vol % of inactive gas, and a space velocity of the raw material is in the range of 500 to 5,000 hr is introduced to the oxidation reactor.

18. The method for producing an acrolein according to claim 15, wherein the shape of the inactive material of the first catalyst layer is a powder shape or a granule shape.

19. The method for producing an acrolein according to claim 15, wherein the first catalyst layer is classified into two catalyst layers, among the two catalyst layers, the catalyst layer in which the complex catalyst particle that is obtained by shaping the mixture of the catalyst effective component material and the granule shape inactive material is filled is disposed at an inlet side of the raw material, the catalyst layer in which the complex catalyst particle that is obtained by shaping the mixture of the catalyst effective component material and the powder shape inactive material is filled is disposed next thereto, and the second catalyst layer in which the pure catalyst particle that is obtained by shaping the catalyst effective component material is filled is disposed next thereto.

20. A method for producing an acrylic acid, the method comprising the steps of:
a) using the oxidation reactor of claim 13 and performing a fixed layer catalyst partial oxidation reaction to propylene to produce an acrolein; and
b) performing a fixed layer catalyst partial oxidation reaction to the produced acrolein.
